(19) United States
(12) Patent Application Publication
(10) Pub. No.: US 2020/0078535 A1
(43) Pub. Date: Mar. 12, 2020

(54) A Blanket for Venipuncture

(71) Applicant: Care Essentials Pty Ltd, Victoria (AU)

(72) Inventors: Abhay Sinha, Victoria (AU); Ishan Sinha, Victoria (AU); Tan Mian, Llantarnam Gwent (GB)

(73) Assignee: Care Essentials Pty Ltd, Victoria (AU)

(21) Appl. No.: 16/606,864

(22) PCT Filed: Apr. 27, 2018
(86) PCT No.: PCT/AU2018/050392

§ 371 Date: Oct. 21, 2019

(30) Foreign Application Priority Data
May 2, 2017 (AU) 2017901598

(51) Int. Cl. A61M 5/42; A61F 7/02

(52) U.S. Cl. CPC A61M 5/427; A61F 2007/0036; A61F 7/02

(57) ABSTRACT

A blanket for venipuncture or cannulation on a patient's hand using forced air warming, including a non-air permeable outer layer and an air permeable inner layer the outer layer and inner layer defined to form an inflatable chamber, the inflatable chamber allowing egress of continuous forced warming air on the patient's hand; wherein in use, continuous forced warming air dilates and allows for visual identification of the patient's hand veins.

### FIELD OF THE INVENTION

The present invention relates to a blanket for venipuncture and other medical procedures.

### BACKGROUND OF THE INVENTION

Conventionally, there is an existing and ongoing problem for medical clinicians, haematologists, anaesthetists and nurses during medical procedures such as venipuncture, to identify and access the veins of patients and more particularly cancer patient's veins inside the patient's elbow or cubital fossa being the median cubital vein or dorsum of the patient's hand including the dorsal metacarpal veins, dorsal venous plexus, cephalic vein and basilic vein. Similar problems exist for accessing the median cubital veins inside the patient's elbow or veins on the dorsum of the patient's hand including the dorsal metacarpal veins, dorsal venous plexus, cephalic vein and basilic vein, the purpose of administering cannula.

Previous attempts to overcome the problem of accessing the median cubital vein or the dorsal metacarpal veins, dorsal venous plexus, cephalic vein and basilic vein on the dorsum of the patient's hand, involved attempting to dilate such veins with a warmed medium such as warmed water. Other attempts involved use of a water bottle or heat pads physically applied to the cubital fossa or the dorsum of the patient's hand, as the case may be.

All of these attempts were not as effective, as they suffered the problems being that once the warming medium was physically removed from the cubital fossa or the dorsum of the patient's hand (as the case may be) the temperature around the cubital fossa or dorsum of the patient's hand dropped and the targeted median cubital vein or the dorsal metacarpal veins, dorsal venous plexus, cephalic vein and basilic vein would then contract to their original position and disappear from sight of the clinician or nurse, before the targeted vein could be accessed for venipuncture, cannulation or other medical purpose.

Another previous attempt to identify and access the veins of patients, including the veins of cancer patients or those patients that fast for a blood test, is to use a medical light torch being an infra-red LED light applied to the cubital fossa or dorsum of the patient's hand. Although this infra-red LED Light does identify the median cubital vein and dorsal metacarpal veins, dorsal venous plexus, cephalic vein and basilic vein, it is an expensive device but always suffers the problem being that the infra-red LED light itself does not provide the necessary dilatation of the vein provided by warming of the median cubital vein and dorsal metacarpal veins, dorsal venous plexus, cephalic vein and basilic vein as described above.

It is therefore an object of the present invention to provide for a device that can identify and dilate the median cubital vein or dorsal metacarpal veins, dorsal venous plexus, cephalic vein and basilic vein for venipuncture or cannulation and to ameliorate the aforementioned problems.

SUMMARY OF INVENTION

Accordingly, in one aspect the present invention provides a blanket for venipuncture or cannulation on a patient's hand using forced air warming, the blanket including:

(a) a non-air permeable outer layer;

(b) an air permeable inner layer contactable to the patient's hand;

(c) the outer layer and inner layer being joined at the periphery of the inner layer to define an inflatable chamber, the chamber allowing egress of the forced warming air through the inner layer over the patient's hand;

wherein in use, continuous forced warming air dilates and allows for visual identification of the patient's hand veins.

In another aspect, the present invention provides a blanket allowing access to a patient's vein for venipuncture or cannulation on the patient's arm using forced air warming, the blanket including:

(a) a non-air permeable outer layer;

(b) an air permeable inner layer contactable to the patient's arm;

(c) the outer layer and inner layer being joined at the periphery of the inner layer to define an inflatable chamber, the chamber allowing egress of continuous forced warming air through the inner layer over the patient's arm;

(d) an access port disposed adjacent to the chamber;

wherein in use, the access port provides access to the patient's arm for venipuncture or cannulation through the access port.

In a further aspect, the present invention provides a method of accessing a patient's vein for venipuncture or cannulation using forced air warming, the method including the following steps:

(a) positioning a blanket over the patient's arm or dorsum hand, the blanket comprising:
(i) a non-air permeable outer layer; and
(ii) an air permeable inner layer contactable to the patient's arm or hand; and
(iii) the outer layer and inner layer being joined at the periphery of the inner layer to define an inflatable chamber, the chamber allowing egress of the forced warming air through the inner layer over the patient's arm or hand; and
(iv) an access port disposed adjacent to the chamber and cubital fossa of the patient's arm; and

(b) applying continuous forced warming air to the arm or hand through the inner layer; and

(c) visually identifying an expanded vein on the arm through the access port or on the hand by lifting the blanket.

Preferably, the inner layer includes an array of perforations.

Preferably, the access port includes a slit.

Preferably, the vein includes any one of the following: median cubital vein; cephalic vein; basilic vein; dorsal metacarpal veins; and dorsal venous plexus.

Preferably, the outer layer and inner layer are integrally formed.

Preferably, the access port is disposed adjacent to a joined portion of the outer layer and inner layer.

One advantage of the blanket for venipuncture or cannulation is that in use, the patient's target median cubital vein or dorsal metacarpal veins, dorsal venous plexus, cephalic vein and basilic vein is dilated and clearly identifiable by the clinician or nurse.

It will be convenient to hereinafter to describe the invention with reference to the following drawings which shows nine (9) preferred embodiments of the blanket for venipuncture according to the invention. The particularity of the attached drawings and following description should not supersede or limit the preceding broad definition of the invention.

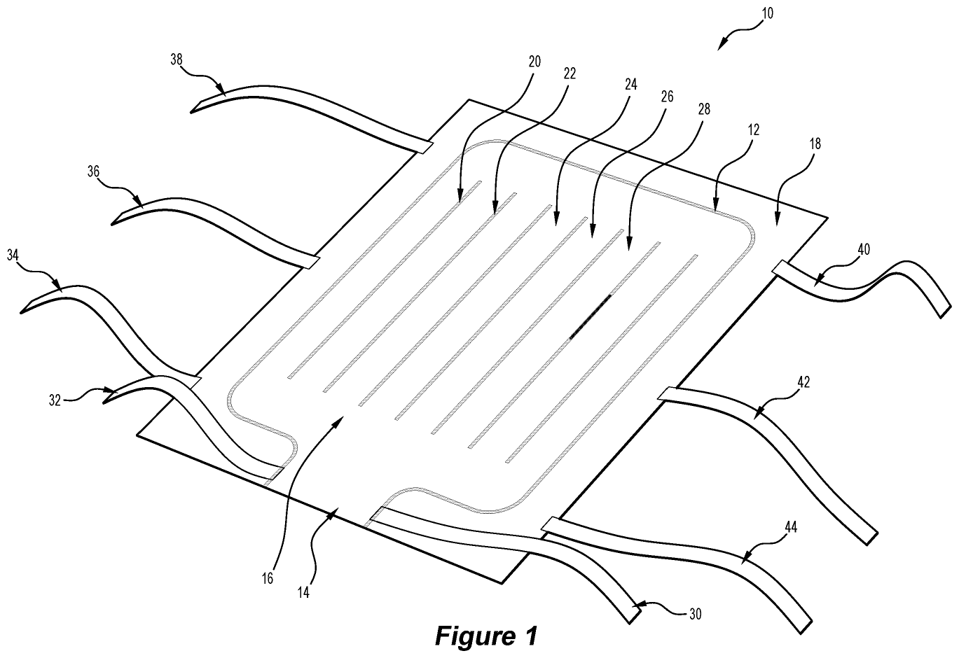

FIG. 1, is a perspective view of a blanket for venipuncture according to a first preferred embodiment of the invention, illustrating the blanket in an unassembled state before use on the patient;

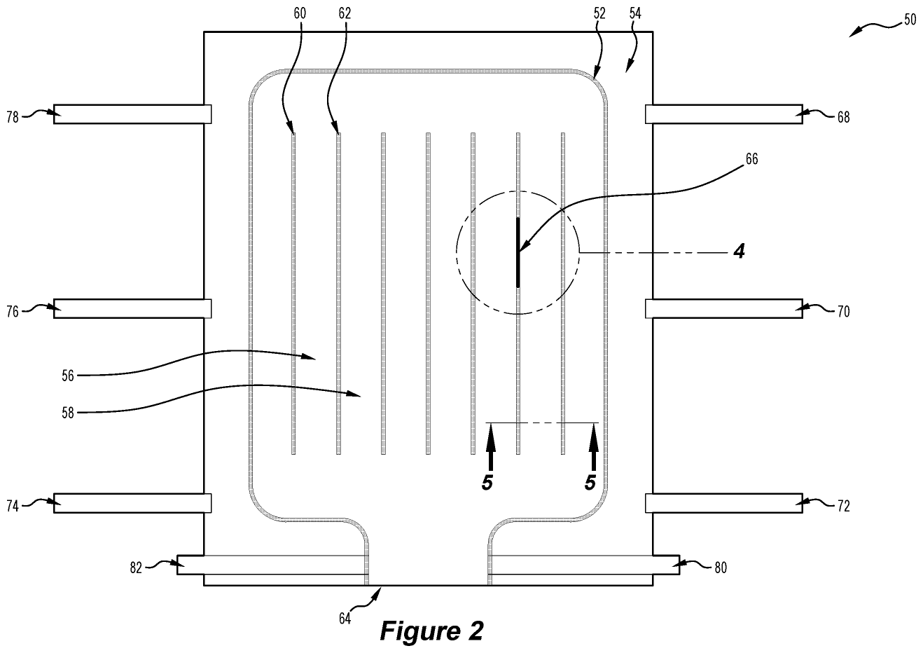

FIG. 2 is a top perspective view of the blanket for venipuncture of FIG. 1, according to a second preferred embodiment of the invention, illustrating the blanket in an unassembled state before use on the patient and the facing outer layer;

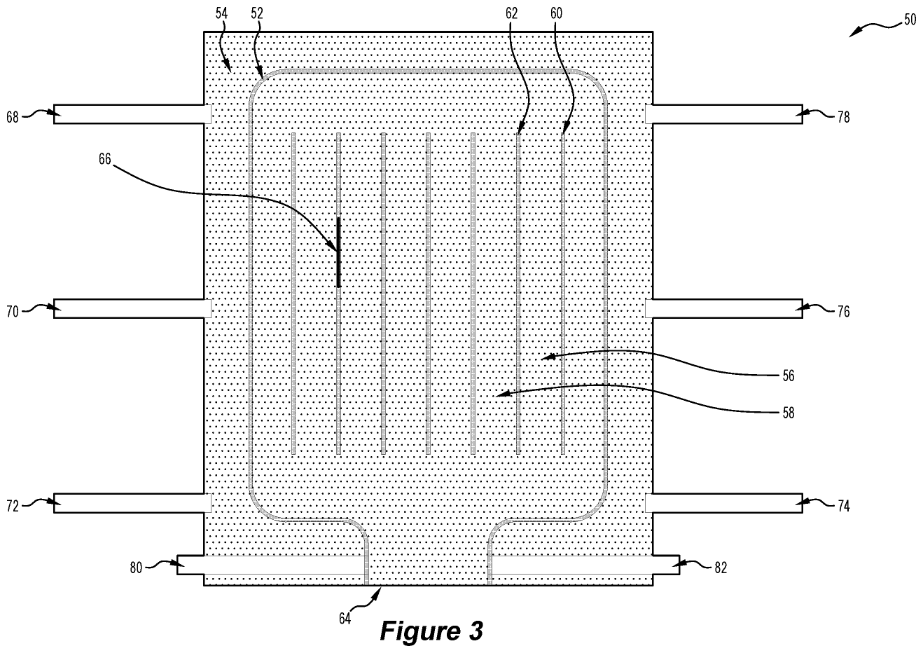

FIG. 3 is a bottom perspective view of the blanket for venipuncture of FIG. 1, according to a third preferred embodiment of the invention, illustrating the blanket in an unassembled state before use on the patient and the facing inner layer;

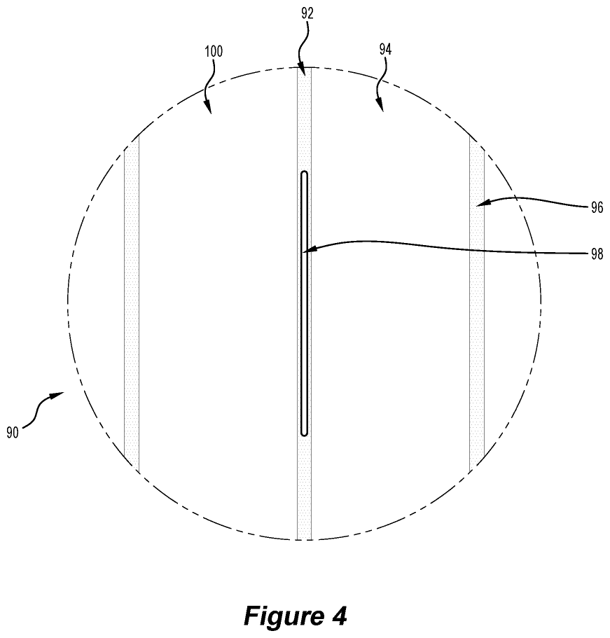

FIG. 4 is an exploded view of the blanket for venipuncture of FIG. 2, taken through circle section “4”, according to a fourth preferred embodiment of the invention, illustrating an access port;

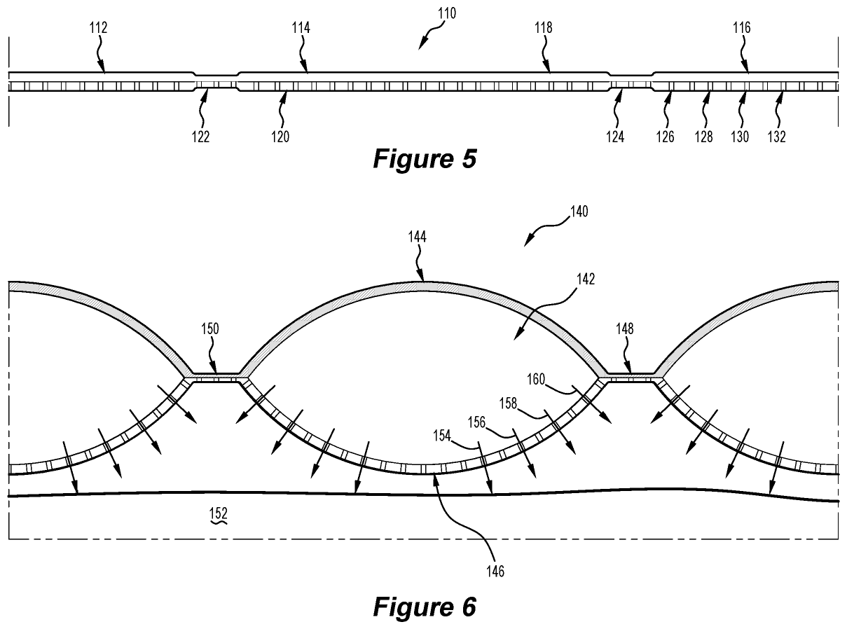

FIG. 5 is a cross sectional view of an inflatable chamber of the blanket for venipuncture of FIG. 2, taken through section 5-5 according to a fifth preferred embodiment of the invention, illustrating, the inflatable chamber with no forced warming air;

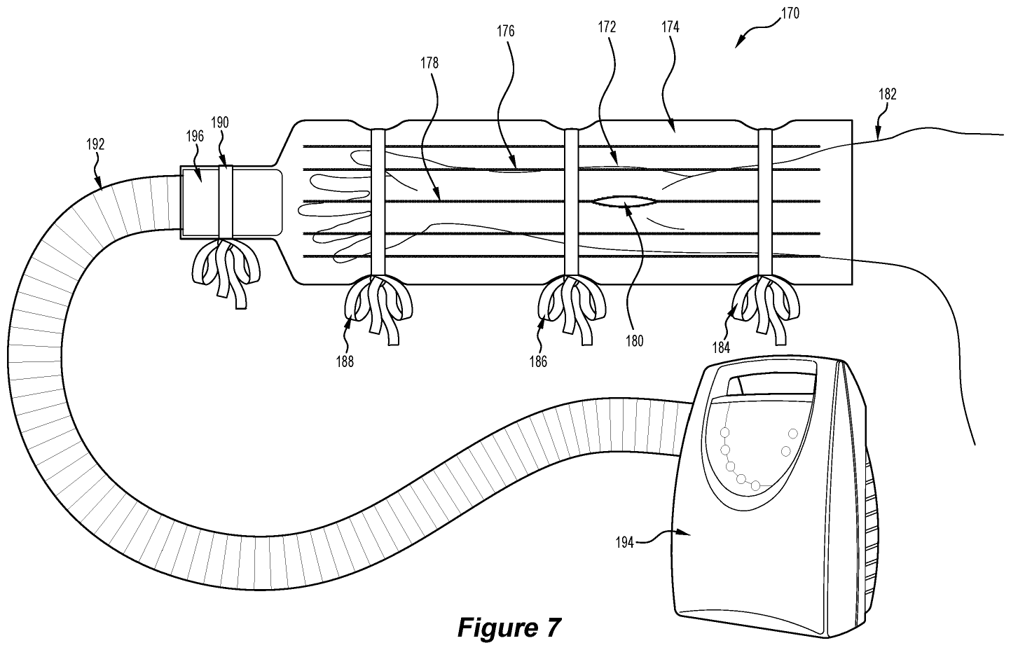

FIG. 6 is a cross sectional view of the inflatable chamber of FIG. 5, according to a sixth preferred embodiment of the invention, illustrating the inflatable chamber in an expanded position egressing forced warming air through the inner layer and onto the patient;

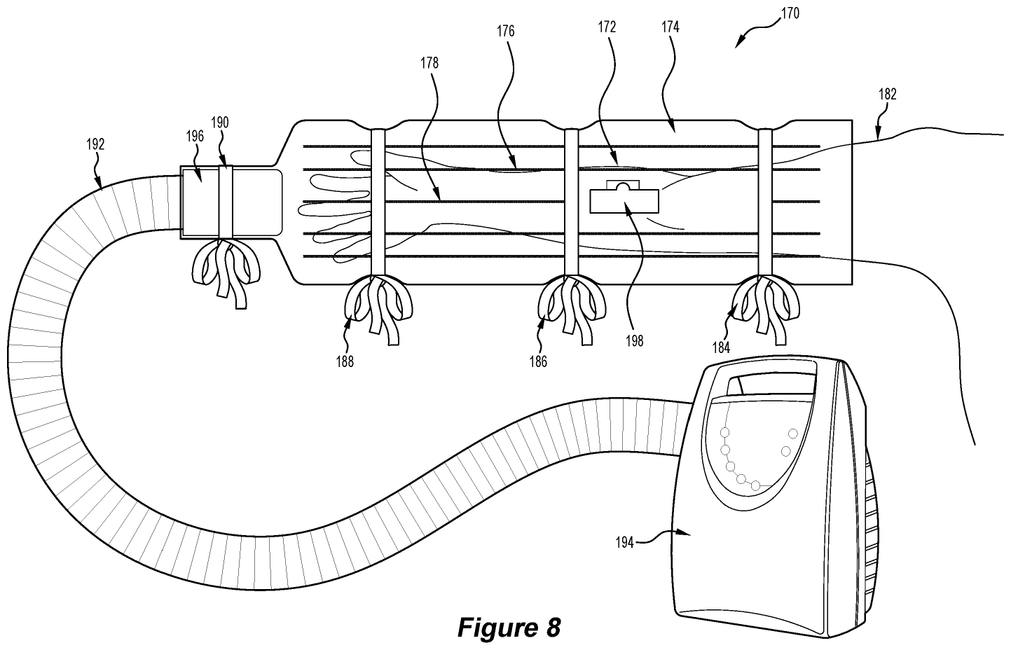

FIG. 7 is a perspective view of the blanket for venipuncture of FIG. 1 used on top of the patient's arm and being connected to a forced warming machine and connectable hose, according to a seventh preferred embodiment of the invention;

FIG. 8 is a perspective view of the blanket for venipuncture of FIG. 1 used on top of the patient's arm and being connected to the forced warming machine and connectable hose, according to an eighth preferred embodiment of the invention;

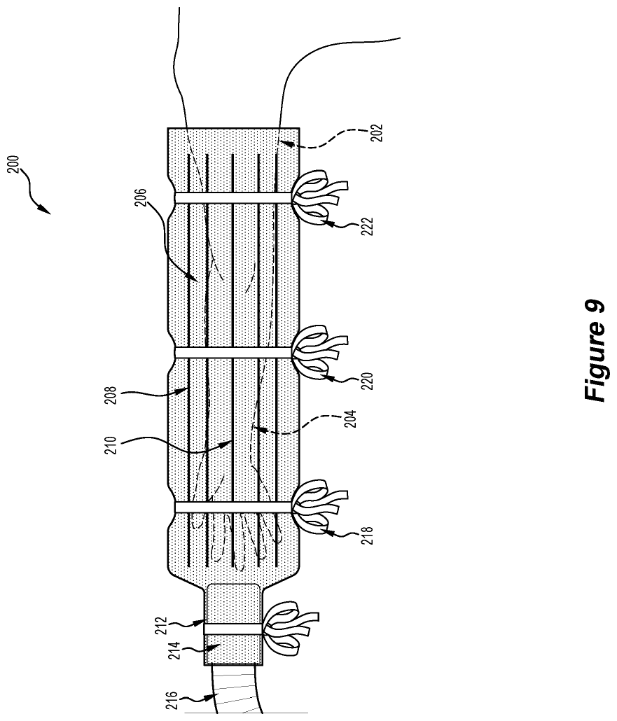

FIG. 9 is a perspective view of the blanket for venipuncture used on top of the patient's arm and being connected to the forced warming machine and connectable hose, according to a ninth preferred embodiment of the invention, illustrating the non-transparent blanket and visual blocking of the patient's arm, as indicated with dotted lines.

### DETAILED DESCRIPTION OF THE INVENTION

FIG. 1, is a perspective view of the blanket for venipuncture 10, illustrating the blanket in an unassembled state before use on the patient, is provided with an inflatable chamber 12. The inflatable chamber 12 is constructed by an outer layer and inner layer being joined at the periphery of the inner layer. The person skilled in the art would appreciate that the inflatable chamber 12 extends not at the absolute outer edge of the inner layer but its periphery. The outer layer is non-air permeable and the inner layer is air permeable. The outer layer presents visually upside toward the medical clinician when the blanket for venipuncture 10 is placed on the patient. The air permeable inner layer is contactable with the patient when the blanket for venipuncture 10 is used. The inflatable chamber 12 is inflatable when receiving forced warming air from any convenient air warming machine currently in use (not shown) though an air inlet 14, the forced warming air which extends into inflatable chamber space 16 that expands the inflatable chamber 12 from its collapsed position.

The non-inflatable portion 18 is not functional in the sense that it does not form part of the inflatable chamber 12, but it is defined by the loose association of the outer layer and inner layer.

The inflatable chamber 12 is defined by joining of the outer layer and periphery of the inner layer or integrally forming of the outer layer and the inner layer of the periphery of the inner layer or thermal welding of the outer layer and inner layer at the periphery of the inner layer.

The blanket for venipuncture 10 is both transparent and non-transparent. That is, when in the transparent form, the user of the blanket for venipuncture 10 being the clinician and/or medical nurse can visually see through the blanket and the entire limbs of the patient that is covered. On the other hand, when in the non-transparent form, the clinician and/or medical nurse cannot visually see through the blanket and the entire limbs of the patient that is covered.

In the transparent form, each of the outer layer and the inner layer are constructed from biaxially orientated polypropylene film, polypropylene film, polyvinyl chloride (PVC) and other medical grade plastic material having a transparent function. It must be stated that biaxially orientated polypropylene film is benign on the human skin and the ideal material for such blankets.

In the non-transparent form, each of the outer layer and the inner layer are constructed from any suitable non-woven material and/or medical grade material having a non-transparent function. The non-woven material or non-woven fabric is benign on the human skin and the ideal material for such blankets.

The air permeable inner layer is constructed with plurality or an array of small apertures or perforations (not shown in FIG. 1) to provide an egress function of the forced warming air from inside the inflatable chamber 12 through the air permeable inner layer and onto the patient for warming to the appropriate body temperature as determined by the clinician and/or nurse. The perforations are evenly spread across the entire surface area of the inner layer such that they operatively apply as a whole, continuous forced air warming over the relevant surface area of the patient. The perforations are of any shape including annular, spherical and square shape.

The inflatable chamber 12 consists of a plurality of interconnected compartments 24, 26 & 28. Specifically, interconnected compartments 24, 26 & 28 which are all disposed or positioned in parallel with each other and are fluted. The person skilled in the art would appreciate that the interconnected compartments 24, 26 & 28 are functionally connected to each other at their respective ends. These interconnected compartments 24, 26 & 28 are formed or defined by joining of a portion of the outer layer and a corresponding portion of the inner layer, or integrally forming of a portion of the outer layer and a corresponding portion of the inner layer or thermal welding of a portion of the outer layer and a corresponding portion of the inner layer to define join 20 and join 22. Alternatively, the join 20 and join 22 is also formed by way of ultrasonic welding a portion of the outer layer and a corresponding portion of the inner layer. Advantageously, the interconnected compartments 24, 26 & 28 functions to provide a uniform and longitudinal distribution of forced warming air over a patient.

Machine ties 30 and 32 serve the function of securing the blanket for venipuncture 10 to a hose of the forced air warming machine (both the hose and forced air warming machine not shown in FIG. 1). The machine ties 30 and 32 are constructed from any suitable material, including cotton, nylon, Velcro™ and any other suitable material. Patient ties 34, 36, 38, 40, 42 and 44, serve the function of securing the blanket for venipuncture 10 to the patient after the blanket for venipuncture 10 is wrapped around the arm or body of the patient (both not shown in FIG. 1). The patient ties 34, 36, 38, 40, 42 and 44 are constructed from any suitable material, including cotton, nylon, Velcro™ and any other suitable material.

Turning to FIG. 2, being the top perspective view of the blanket for venipuncture 50, having the inflatable chamber 52, the inflatable chamber 52 in turn, includes a plurality of interconnected compartments 56 and 58 separated by join 60 and join 62 respectively. The non-inflatable portion 54 is defined by the loose association of the outer layer and inner layer.

The join 60 and join 62 are created by joining a portion of the outer layer and a corresponding portion of the inner layer, or integrally forming of a portion of the outer layer and a corresponding portion of the inner layer or thermal welding of a portion of the outer layer and a corresponding portion of the inner layer. Alternatively, the join 60 and join 62 are each formed by way of ultrasonic welding a portion of the outer layer and a corresponding portion of the inner layer.

Air inlet 64 extends into or articulates with the inflatable chamber 52 such that when forced warming air is blown through the air inlet 64 and into the inflatable chamber 52 when the blanket for venipuncture 50 is in a lay-flat orientation and the forced warming air is blown substantially parallel with the interconnected compartments 56 & 58.

An access port 66 is disposed adjacent to the inflatable chamber 52 which provides physical access to the patient through the blanket for venipuncture 50 described further by FIG. 4 below.

Patient ties 68, 70, 72, 74, 76 & 78 serve the function of securing the blanket for venipuncture 50 to the patient (not shown) after the blanket for venipuncture 50 is wrapped around the arm or body of the patient (both not shown in FIG. 2). Machine ties 80 and 82 serve the function of securing the blanket for venipuncture 50 to a hose of the forced air warming machine (both not shown in FIG. 2).

Turning to FIG. 3 is a bottom perspective view of the blanket for venipuncture 50 of FIG. 1, illustrating the blanket for venipuncture 50 in an unassembled state before use on the patient and the facing inner layer. The blanket for venipuncture 50 has the inflatable chamber 52 and a plurality of interconnected compartments 56 and 58 therethrough, separated by join 60 and join 62 The non-inflatable portion 54 is defined by the loose association of the outer layer and inner layer. The joins 60 and join 62 are created by joining a portion of the outer layer and a corresponding portion of the inner layer, or integrally forming of a portion of the outer layer and a corresponding portion of the inner layer or thermal welding of a portion of the outer layer and a corresponding portion of the inner layer. Alternatively, the join 60 and join 62 is also formed by way of ultrasonic welding a portion of the outer layer and a corresponding portion of the inner layer.

The air inlet 64 extends into or articulates with the inflatable chamber 52 such that when forced warming air is blown through the air inlet 64 and into the inflatable chamber 52 when the blanket for venipuncture 50 is in a lay-flat orientation and the forced warming air is blown substantially parallel with the interconnected apartment 56 and interconnected apartment 58.

An access port 66 is disposed adjacent to the inflatable chamber 52 which provides physical access to the patient through the blanket for venipuncture 50 described further by FIG. 4 below.

Patient ties 68, 70, 72, 74, 76 & 78 serve the function of securing the blanket for venipuncture 50 to the patient (not shown) after the blanket for venipuncture 50 is wrapped around the arm or body of the patient (both not shown in FIG. 3). Machine ties 80 and 82 serve the function of securing the blanket for venipuncture 50 to a hose of the forced air warming machine (both not shown in FIG. 3).

The person skilled in the art would appreciate by considering FIG. 3. that the inner layer has spotted shading, which illustrates the perforations of the inner layer. These perforations provide the necessary egress function of the forced warm air to pass through the inflatable chamber 52 and through the perforations defined within which is further described by FIG. 5 below.

Referring to FIG. 4 is an exploded view of the blanket for venipuncture of FIG. 2, by way of circle section “4” 90, having a join 92, formed by joining, integrally forming, or thermally welding together a portion of the outer layer and corresponding portion of the inner layer or alternatively formed by ultrasonic welding of a portion of the outer layer and corresponding portion of the inner layer. The join 92 is provided with a slit 98, which is selectively openable, through a perforated tear strip, taping, folding, sealing, snapping, or a combination thereof (not illustrated in this FIG. 4). The slit 98 is initially sealed closed and is reversibly openable. The join 92 and join 96 are separated by interconnected compartments 94 and 100. The slit 98 is advantageously positioned along the join 92 such that the inflatable chamber (not illustrated) can still function as the interconnected compartment 94 and interconnected compartment 100 are still functional and not perforated by the slit 98.

Turning to FIG. 5 is a cross sectional view of the inflatable chamber 110 of FIG. 2, taken through section 5-5, illustrating the interconnected compartments 112, 114 and 116 all in a flat and un-used state with no forced warming air passing therethrough. The outer layer 118 is flexible and impermeable to air engages the inner layer 120 which is also flexible and is air permeable. Perforations 126, 128, 130 and 132 disposed within the inner layer 120 provide respective air channels through the entire inner layer 120. The join 122 and join 124 are each defined by joining a portion of the outer layer 118 and a corresponding portion of the inner layer 120, or integrally forming of a portion of the outer layer 118 and a corresponding portion of the inner layer 120 or thermal welding or ultrasonic welding of a portion of the outer layer 118 and a corresponding portion of the inner layer 120.

It will be appreciated by the person skilled in the art that the inflatable chamber 110 can use both transparent and non-transparent material both having the same air permeable perforations 126, 128, 130 and 132 disposed within the inner layer 120.

Turning to FIG. 6 is the cross-sectional view of the inflatable chamber 140 of FIG. 5 illustrating the inflatable chamber 140 in an expanded position egressing forced warming air through the inner layer 146 and onto a patient 152. The interconnected compartment 142 has flexibly expanded by way of forced warming air passing therethrough. The outer layer 144 being flexible and impermeable to air, flexibly expands as well as the inner layer 146 which is also flexible, but is air permeable. Egressed air 154, 156, 158 and 160 through corresponding perforations (unnumbered) disposed within the inner layer 146 provide the air channel through the entire inner layer 146 and onto the patient 152 (in part) including a dorsum hand or arm of the patient 152. The join 148 and join 150 are each sufficiently bonded or joined together to provide the necessary structure of the interconnected compartment 142 during the forced warming air egressed state.

By considering Figure, 3, FIG. 5 and FIG. 6, the person skilled in the art would appreciate that the inner layer has an array of perforations throughout the entire surface area of the inflatable chambers 52, 110 and 140 respectively, which provides the necessary egress function of the forced warming air through the inner layer 120 of FIG. 5 and inner layer 146 of FIG. 6 and onto the patient 152.

Now FIG. 7 is a perspective view of the blanket for venipuncture 170 used on top of the patient's arm 182 and being connected to the forced air warming machine 194 and hose 192 which channels the forced warming air to the blanket for venipuncture 170 through the air inlet 196 and through the interconnected compartments 172 and 174 situated parallel with each other and are fluted. The join 176 and join 178 separate the interconnected compartments 172 and 174 are previously described herein.

The person skilled in the art will appreciate that in use, the interconnected compartments 172 and 174 are disposed substantially coaxially with the patients arm 182 when the blanket for venipuncture 170 is used. The person skilled in the art will also appreciate that FIG. 7 illustrates the transparent version of the blanket for venipuncture 170 such that the patient's arm 182 is clearly transparent through the blanket for venipuncture 170, at all times during use by the clinician or medical nurse (not illustrated in this FIG. 7). A non-transparent blanket is used and also described herein. The person skilled in the art will further appreciate that FIG. 7 illustrates that the blanket for venipuncture 170 in use is placed on top of the patient's arm 182. This means that in use, the blanket for venipuncture 170 can be lifted up momentarily above the dorsal hand near the air inlet 196 such the dorsal hand can be accessed for cannulation by the medical practitioner of the patient's veins, being the dorsal metacarpal veins, dorsal venous plexus, cephalic vein and basilic vein.

The blanket for venipuncture 170 includes an access port 180 which allows the clinician, anaesthetist and nurse to visually identity and physically access the patients arm 182 and perform medical treatment (including venipuncture or the administration of cannula) on the patient's arm 182 through the access port 180. The patient's arm 182 is positioned or disposed adjacent or situated in a manner whereby the patient's cubital fossa and therefore median cubital vein is accessible through the access port 180. Other patient's veins are also accessible near the cubital fossa, include the cephalic vein and the basilic vein. The patient ties 184, 186 and 188 are illustrated in use which are assembled by hand tying by the clinician, anaesthetist and nurse when assembling or securing the blanket for venipuncture 170 to the patient's arm 182.

In normal use, the blanket for venipuncture 170 can be used in a variety of ways. Firstly, the blanket for venipuncture 170 is wrapped around the patient's arm 182 and tied using the patient ties 184, 186 and 188 by the clinician's hands (not shown). The hose 192 of the forced air warming machine 194, is then inserted into the air inlet 196 and tied together by the machine tie 190, by tying over the air inlet 196 end of the blanket for venipuncture 170. The forced air warming machine 194 is then turned on by the clinician or nurse and then continuous forced warming air then passes through the hose 192 and the air inlet 196 and expands the inflatable chamber (not numbered) by forced warming air passing through the interconnected compartments 172 and interconnected compartments 174 and further through the inner layer (not numbered) as described above and warming the patient's arm 182 with continuous forced warming air. Secondly and alternatively, the blanket for venipuncture 170 is placed on top of the patient's arm 182 without the need for using the patient tie 184, patient tie 186 and patient tie 188.

In an alternative embodiment and which is not illustrated in FIG. 7, the blanket for venipuncture 170 does not include the patient tie 184, but is optionally replaced with a tourniquet. The tourniquet is made of any suitable elasticated material and is positioned proximal or at the upper end of the patient's arm 182. The tourniquet is fixed or engageable with the blanket for venipuncture 170.

Turning to FIG. 8 is effectively the same as FIG. 7, being the blanket for venipuncture 170 except with the addition of a tear strip 198 covering the access port 180 (of FIG. 7) which is not illustrated in this FIG. 8, but in use is visually identifiable or alternatively not visually identifiable, as the tear strip 198 is transparent or non-transparent air non-permeable material. The tear strip 198 may also have a taping, folding, sealing, snapping function or a combination thereof. The clinician, anaesthetist and nurse in use, can visually identify the patient's arm 182 through the tear strip 198 and then physically accesses the patient's arm 182 by opening the tear strip 198 with their hand and pass a medical instrument including a blood syringe for venipuncture or cannula for cannulation through the access port 180 (of FIG. 7) and physically contact the patient's cubital fossa and median cubital vein, cephalic vein and basilic vein.

The access port 180 of FIG. 7 and FIG. 8 having the tear strip 198, is unique and provides the access function to the patient's arm veins through the blanket for venipuncture 10, 50 and 170 of FIGS. 1 to FIG. 3, and FIG. 7 to FIG. 8, whilst undertaking continuous forced air warming.

The access port 180 provides the access to the patient's cubital fossa, which provides access to the median cubital vein, the cephalic vein and basilic vein of the patient's arm for venipuncture or cannulation.

Finally, turning to FIG. 9 illustrates a perspective view of the blanket for venipuncture 200 used by placing on top of the patient's arm 202 and patient's arm 204 and being connected to the forced warming machine (not shown) and connectable hose 216 (shown in part) illustrating the non-transparent blanket (as indicated by shading) and visual concealment of the patient's arm 202 and patient's arm 204 (as indicated by dotted lines). It will be appreciated by the person skilled in the art that the blanket for venipuncture 200 does not include an access port in FIG. 9, although it is optionally contemplated. An interconnected compartment 206 is defined by join 208 and join 210 formed as described herein. A filter cap 212 provides an air filter for bacteria, fungi and viruses connected to a hose 216 for forced warming air (shown in part), the filter cap 212 being inserted through the air inlet 214. Patient tie 218, patient tie 220 and patient tie 222 is used optionally to secure the blanket for venipuncture 200 around the patient's hand and patient's arm 202 and patient's arm 204. The person skilled in the art will appreciate that the blanket for venipuncture 200 can be placed on top of the patient's hand and patient's arm 202 and patient's arm 204 without using patient tie 218, patient tie 220 and patient tie 222.

The unique feature of the blanket for venipuncture 10, 50, 170 and 200 is the use and application of continuous forced air warming in clinical use that dilates the veins on the dorsal hands of patients or the veins on the cubital fossa on the patient's arm, such that they are all clearly clinical identifiable for access by way of venipuncture and/or cannulation by the clinician and/or medical nurse.

All of the above embodiments FIG. 1 to FIG. 9 of the blanket for venipuncture 10, 50, 170 and 200 are single use disposable blankets. Once the blanket for venipuncture 10, 50, 170 and 200 are used on a single patient they are destroyed so as to avoid any potential cross-infection by using on subsequent patients. This reduces cleaning and laundry costs for medical clinics and hospitals.

It will also be appreciated by the person skilled in the art that the blanket for venipuncture 10, 50, 170 and 200 are still functional even without using the relevant patient ties 34, 36, 38 40, 42 and 44 (of FIG. 1 as a non-limiting example only). The blanket for venipuncture 10 can be wrapped under the patient's arm, with no need for tying of the patient ties 34, 36, 38 40, 42 and 44. The blanket for venipuncture 10 can still be inflated providing continuous forced warming air to the patient's arm. Importantly, the blanket for venipuncture 10, 50, 170 and 200 can be placed on top of the patient's arm and provide continuous forced air warming without the need for tying of the patient ties 34, 36, 38 40, 42 and 44 of FIG. 1 or wrapping around the patient's arm 202 and 204 of FIG. 9 for example. Notwithstanding FIG. 9, the blanket for venipuncture 200 does not need to be wrapped around the patient's arm 202 and patient's arm 204 but can be placed on top thereof, without using the patient ties 218, 220 and 222. This has the advantage for the medical clinician, haematologist, anaesthetist or nurse to visually and physically access the patient's entire arm by momentarily lifting the blanket during use.

By referring to FIG. 7 and FIG. 8, the method of accessing a patient's arm 182 vein for venipuncture or cannulation using continuous forced air warming, includes positioning the blanket for venipuncture 170 over or on top the patient's arm 182. The blanket for venipuncture 170 has already been described herein. The access port 180 is positioned adjacent to the cubital fossa of the patient's arm 182. Optionally, the patient ties 184, 186 and 188 are used by the nurse or clinician to secure the wrapped blanket for venipuncture 170 around the patients arm 182. The forced air warming machine 194 is secured to the blanket for venipuncture 170 by inserting the hose 192 into the air inlet 196 and secured by the machine tie 190. Forced warming air is continuously applied to the dorsum of the patient's hand and/or the patient's arm around the cubital fossa through the inner layer of the blanket for venipuncture 170 from the forced air warming machine 194.

As continuous forced warming air is applied to the dorsum of the patient's hand and/or the patient's arm around the cubital fossa region, both through the blanket for venipuncture 170, the nurse or clinician visually identifies an expanded vein within the cubital fossa through the access port 180 by opening the tear strip 198. The heat expanded vein is accessed and identified through the access port 180 by pulling the tear strip 198 (of FIG. 8) for venipuncture. Other medical administration includes cannulation. Further the veins on the dorsum hand being the dorsal metacarpal veins and dorsal venous plexus and even the cephalic vein; basilic vein on or adjacent the dorsum hand are expanded by continuous forced air warming and accessed by lifting the blanket for venipuncture 170 at the air inlet 196 end, momentarily exposing the dorsum hand.

One advantage of the blanket for venipuncture or cannulation is that in use, the patient's target median cubital vein or dorsal metacarpal veins, dorsal venous plexus, cephalic vein and basilic vein is dilated and clearly identifiable by the clinician or nurse.

Various alterations and/or additions may be made to the blanket for venipuncture, hereinbefore described in this Specification, without departing from the spirit, ambit or scope of the invention.

A reference to any prior art in this Specification is not, and should not be taken as, an acknowledgment or any form or suggestion that the prior art forms part of the common general knowledge.

1-39. (canceled)

40. A blanket for venipuncture or cannulation on a patient's hand using forced air warming, the blanket including:
(a) a non-air permeable outer layer;
(b) an air permeable inner layer contactable to the patient's hand;
(c) the outer layer and inner layer being joined at the periphery of the inner layer to define an inflatable chamber, the chamber allowing egress of the forced warming air through the inner layer over the patient's hand;
wherein in use, continuous forced warming air dilates and allows for visual identification of the patient's hand veins.

41. The blanket according to claim 40, wherein the inner layer includes an array of perforations.

42. The blanket according to claim 40, wherein the outer layer and inner layer are integrally formed.

43. The blanket according to claim 40, wherein the outer layer and inner layer are thermally or ultrasonically welded together.

44. The blanket according to claim 40, wherein the inflatable chamber includes a plurality of interconnected compartments.

45. The blanket according to claim 44, wherein each one of the interconnected compartments are disposed in parallel.

46. The blanket according to claim 44, wherein each one of the interconnected compartments are fluted.

47. The blanket according to claim 44, wherein each one of the interconnected compartments are defined by thermally or ultrasonically welding together a portion of the outer layer and a corresponding portion of the inner layer.

48. The blanket according to claim 40, further including an inlet port for receiving forced air warming into the inflatable chamber.

49. The blanket according to claim 40, including at least one first tie for securing to a forced air machine.

50. A blanket allowing access to a patient's vein for venipuncture or cannulation on the patient's arm using forced air warming, the blanket including:
(a) a non-air permeable outer layer;
(b) an air permeable inner layer contactable to the patient's arm;
(c) the outer layer and inner layer being joined at the periphery of the inner layer to define an inflatable chamber, the chamber allowing egress of continuous forced warming air through the inner layer over the patient's arm;
(d) an access port disposed adjacent to the chamber, the access port including a slit;
wherein in use, the access port provides access to the patient's arm for venipuncture or cannulation through the access port.

51. The blanket according to claim 50, wherein the outer layer and inner layer are integrally formed or the outer layer and inner layer are ultrasonically welded together.

52. The blanket according to claim 50, wherein the inflatable chamber includes a plurality of interconnected compartments, wherein each one of the interconnected compartments are disposed in parallel.

53. The blanket according to claim 52, wherein each one of the interconnected compartments are fluted and each one of the interconnected compartments are disposed substantially coaxially with the patient's arm.

54. The blanket according to claim 52, wherein each one of the interconnected compartments are defined by thermally or ultrasonically welded together a portion of the outer layer and a corresponding portion of the inner layer.

55. The blanket according to claim 50, including at least one first tie for securing to a forced air machine.

56. The blanket according to claim 50, including a plurality of second ties for securing to the patient.

57. The blanket according to claim 50, wherein the use is a single use.

58. The blanket according to claim 50, wherein the blanket is transparent or non-transparent.

59. The blanket according to claim 50, wherein the outer layer and the inner layer are both constructed from any one or a combination of: biaxially orientated polypropylene film; polypropylene film; non-woven material; polyvinyl chloride; and other medical grade plastic.
